(19) United States
(12) Patent Application Publication
(10) Pub. No.: US 2009/0012586 A1
(43) Pub. Date: Jan. 8, 2009

(54) Portable wearable cold laser hair growth stimulator and skin treatment system

(76) Inventor: Lawrence Eliezer Kepecs, Wesley Hills, NY (US)

Correspondence Address: Lawrence Eliezer Kepecs, 4 Fieldcrest Drive, Wesley Hills, NY, 10952, US

(21) Appl. No.: 11/900,600

(22) Filed: Sep. 13, 2007

Related U.S. Application Data
(60) Provisional application No. 60/958,259, filed on Jul. 5, 2007.
(60) Provisional application No. 60/965,995, filed on Aug. 24, 2007.

(51) Int. Cl. A61B 18/20

(52) U.S. Cl. 607/89

(57) ABSTRACT

A portable, wearable, therapeutic hair, scalp, skin, and hearing treatment system for: reducing hair loss in humans; treating dandruff, loss of hair color, increasing hair tensile strength, stimulating new hair growth, and hearing restoration by supplying laser light from a Light Emitting Diode (LED) to the affected area by means of a hat, or in conjunction with a topical Minoxidil-Finasteride mixture and Ketoconazole shampoo. The device also aids in the therapeutic healing of many different skin disorders, and chronic conditions. The lasers are adjustable, focusing on the affected areas requiring treatment. The device slips into a mirrored base containing a flat clear glass and reflective surface for ease in treating other skin disorders. The body treatment device contains a portable “sit-in chamber”. The unique snap in or screw in lasers or LED's can be easily changed altering the desired wavelength to treat a plethora of skin disorders.

This non-provisional utility patent application is a Continuation-In-Part of the singularly owned provisional application entitled “Portable Wearable Cold Laser Hair Growth Stimulator,” filed on Jul. 5, 2007, bearing U.S. Ser. No. 60/958,259 and filed again Qn Aug. 24, 2007, bearing U.S. Ser. No. 60/965,995 and naming Lawrence Eliezer Kepecs, the named inventor herein, as sole inventor, the contents of which is specifically incorporated by reference herein in its entirety which was a non-provisional continuation of the singularly owned provisional application entitled “Portable Wearable Cold Laser Hair Growth Stimulator,” filed Jul. 5, 2007, bearing U.S. Ser. No. 60/958,259 and filed again on Aug. 24, 2007, bearing U.S. Ser. No. 60/965,995 and naming Lawrence Eliezer Kepecs, the named inventor herein, as sole inventor, the contents of which is specifically incorporated by reference herein in its entirety.

### BACKGROUND OF THE INVENTION

1. Technical Field

The present invention relates to a method and apparatus for increasing hair tensile strength, and treating alopecia, hair loss, loss of hair color (graying), dandruff, hearing loss, Tinnitus, Vertigo, eczema, psoriasis, wrinkles, gum recession, skin burns and wounds, some cancers, acne, enlarged pores, oily skin, stretch marks, Folliculitis, Arthritis, back pain, Bursitis, Carpal Tunnel Syndrome, headaches, neck pain, repetitive stress injuries, TMJ pain, tendonitis, sprains, swelling, excessive perspiration, viral warts, precancerous solar keratosis, sun damaged skin, skin ulcers, cellulite, damaged DNA due to environmental exposure to the sun, sunburn, insomnia, jet lag, immune dysfunction disorders, aging, and stimulate collagen. In particular, it relates to a method of treating the scalp, hair, skin and hearing of an individual to increase the blood flow and general health of the skin to promote the healthier growth of hair and restoration of hair color, reduction and elimination of dandruff, increasing hair tensile strength, and may also therapeutically aid in the healing of the above mentioned problems.

2. Background Art

The loss of hair and hearing has traditionally been a problem for a substantial percentage of the population. Whether the problem is alopecia (male pattern baldness) or thinning hair, or damage to the inner ear's delicate cilia due to loud noises or age, the individuals affected will generally find this to be distressing and detrimental to their appearance and/or hearing function. In addition, the loss of hair will often make individuals appear to be older than they are. For this reason, a variety of attempts have been made to improve an individual's appearance by restoring the appearance of a full head of hair.

Early attempts to address this problem focused on the use of hairpieces (i.e., toupees or wigs). While these devices provided some degree of success, they have several drawbacks. In particular, they often have an unnatural appearance, which allows them to be easily detected, even from a distance, by other individuals. In order to make one of these devices appear to be the natural hair of the wearer, they must be constructed in a fairly expensive manner using real human hair, which is matched in color to the individual's remaining hair. Unfortunately, many individuals cannot afford a high quality hairpiece such as this, and settle for a lower quality hairpiece fabricated from synthetic fibers.

In addition, hairpieces have another drawback in that they may be inconvenient to use in situations, such as swimming, where they may become damaged or loosened. In this situation, the individual may become embarrassed due to the failure of the device. It would be desirable to improve the appearance of an individual's hair without having to resort to hairpieces fastened to an individual's head, which are expensive and occasionally prone to failure due to environmental circumstances.

In the case of hearing aids, they too may be inconvenient to use in situations, such as swimming where they will become damaged. Some individuals are also self-conscious about their appearance with a hearing aid. Those skilled in the art of hair restoration will recognize that more complicated mechanical solutions such as “hair weaving” exist. These more complicated solutions typically have the same drawbacks and problems as those associated with hairpieces. In addition, they are often more expensive than conventional hairpieces.

Another attempt to address this problem has been to surgically replace missing hair with “hair plugs.” This surgical solution overcomes the problems created by the use of hairpieces in that the replacement hair plugs use the real hair of the individual, which results in a perfect color match and a natural appearance. In addition, the individual has no restrictions, such as those discussed above in regard to swimming and other activities, which were discussed in regard to hairpieces. Unfortunately, this method of treating hair loss is expensive, often not covered by health insurance, and requires the use of medical professionals for the surgical hair transplant procedure. As a result, this procedure may not be available to a substantial portion of the public due to its high cost. It would be desirable to have a method of facilitating the growth of hair, which was economically available to a substantial part of the entire public.

In addition to hairpieces and surgical transplants, pharmaceutical products have also been developed to encourage hair growth. These products can take the form of ingestible medications or topical skin treatments. The ingestible medications have been proven to encourage hair growth, but they have several significant drawbacks. In particular, they are typically prescription medications which require the cost and inconvenience of visiting a physician to obtain a prescription. In addition, the fact that they are prescription medications typically means that they will have a higher cost than non-prescription drugs. Perhaps, more important than the issue of cost is the potential side effects of ingestible drugs. Quite often, the use of this type of ingestible medications may result in serious health side effects, such as damage to the individual's liver, or other internal organs, or present other serious side effects. It would be desirable to have a method of stimulating hair growth, which did not carry the risks of side effects inherent in ingestible pharmaceutical medications.

Another type of pharmaceutical medication has been the use of topical skin treatments. This type of medication is often similar to prescription medications with the same cost disadvantage of ingestible medications. While some are now available as over-the-counter preparations, they typically have a reduced strength and are less effective than are their prescription counterparts. In addition, they typically have to be applied every day to achieve and maintain their desired results. It would be desirable to have an effective low-cost method of stimulating hair growth, which did not require potential visits to a physician, a continuous use of expensive medications, and daily treatments to ensure results. The method contained herein does not require medications, but recommends for at least the first year, to apply the unique Minoxidil-Finasteride mixture (ie: solution, suspension, or emulsion, alone or in combination or in other forms common or known to those skilled in art of topical and oral delivery), and a Ketoconazole shampoo to help expedite results.

Scientists in Europe and Asia have found over the last 25 years that lasers can be used to stimulate hair growth. Devices have been developed having structures similar to a large floor mounted, or chair mounted, helmet. These devices contain multiple laser assemblies, and are designed to irradiate the individual's entire scalp and hair with laser energy. It has been found that there are several disadvantages associated with this approach. In particular, these are typically very large and expensive commercial devices, which are found in beauty salons and spas. In order to take advantage of them, an individual will go to the establishment where the devices are located and pay for treatments on a per treatment basis. Over time, this represents a fairly expensive proposition for the individual, and typically requires a trained operator to be present and to conduct the treatment.

It would be desirable to have an inexpensive method of applying laser treatments which does not require an individual to go to a specific location where large laser devices are used, which does not require the individual to pay every time a laser treatment is taken, and which maximizes the amount of laser energy applied to the scalp. Likewise, it would be desirable to have a laser treatment device, which has a relatively small, and compact number of lasers, but equally as effective and which could apply laser energy to the individual's scalp.

Another problem associated with hair is the loss of hair color (i.e., greying) that has the effect of making an individual appear to be older. Individuals often attempt to treat this problem by dying their hair. Unfortunately, this method of treatment has several disadvantages. For example, as was the case with hairpieces, discussed above, it can be difficult to produce the proper hair color, which may make it obvious that the hair was dyed. In addition, the dyed hair tends to fade over time, which results in re-appearance of the grey hair and a persistent change in color. Individuals who dye their hair typically have to re-dye their hair periodically. Of course, this represents an ongoing expense and inconvenience to the individual. Further, unless the dying process is performed by a trained individual, the results may be undesirable and unattractive. This further increases the cost of hair dying due to the need to hire a trained professional to perform the process. It would be desirable to have a method of treating loss of hair color without having to have an ongoing expense for dyes, or an ongoing expense for trained professionals to apply the dyes, and an ongoing inconvenience.

It has also been found that light such as LED's, while lacking the characteristics of power concentration inherent in laser light can provide stimulation to the scalp, which has beneficial and therapeutic effects in terms of stimulating the natural growth of hair. Unfortunately, the same problems encountered when attempting to deliver laser light to the scalp are also found when delivering LED non-coherent light. In particular, the problems associated with large stationary devices found in hair salons and spas, as well as the inability to penetrate an individual's hair would be found in either category of light emitting device, so it is recommended when using this new device, to cut the hair short for the treatments, so that the light can penetrate the scalp.

While providing several methods of treating alopecia, hair loss, and greying, the prior art has failed to provide an apparatus which is inexpensive to manufacture, has a minimum number of components, and can be used without leaving the individual's home or using costly commercial equipment and trained personnel.

The following study by John Satino and Michael Markou, and reported by the International Journal of Cosmetic Surgery and Aesthetic Dermatology—Volume 5, Number 2, 2003 will be quoted here regarding low level laser therapy for hair. The authors wished to confirm the efficacy of low level laser therapy (LLLT) using a Hair-Max Laser Comb for the stimulation of hair growth and also to determine what effect LLLT with this device had on the tensile strength of hair. Thirty-five patients, 28 males and 7 females, with androgenetic alopecia (AGA) underwent treatment for a six-month period. Both the hair counts and tensile strength of the hair were affected very beneficially in both sexes in the temporal and vertex regions, with the males' vertex areas showing the most improvement.

### Introduction to the Study

ALTHOUGH LOW-LEVEL LASER THERAPY (LLLT), the therapeutic application of low-energy lasers to medicine, has been used for photobiostimulation for more than thirty years now, in the past it has primarily been used to accelerate the healing of burns or wounds, or alternatively, to ease or relieve pain. Furthermore, LLLT has gained credibility and common usage in some parts of the world, such as Japan, the Scandinavian countries, and Australia, while in other parts of the world, such as North America, a lack of recognition of its efficacy has remained. Even though there are more than 2,500 papers related to LLLT in the scientific literature, (1) only one printed reference, Professor Pekka Pontinen's text, was found which actually discussed the use of LLLT “to stimulate hair growth.”₍₂₎ Even in this source, the information was limited to one paragraph, which refers to one paper given in Sorrento in 1982 which reported increased hair growth after LLLT in animals,₍₃₎ and a foreign language publication in 1983 which reported favorable results with LLLT in the treatment of Alopecia areata.₍₄₎ The authors heard about Dr. Martin Unger's paper in Puerto Vallarta, Mexico, discussing the use of LLLT for hair biostimulation,₍₅₎ and around this same time period, the fall of 2001, became interested in the HairMax LaserComb (Lexington International, Boca Raton, Fla.), secondary to several anecdotal reports that they had heard about this device. It was at this time that the authors decided to carry out their own study to determine whether the Hair-Max LaserComb was effective with regard to stimulating hair growth. The authors also wished to determine what affect LLLT had on the tensile strength of hair and undertook to determine this during the same study.

### Materials and Methods of the Study

The HairMax LaserComb was selected as the LLLT device for many important reasons. As noted above, the authors had heard several favorable anecdotal reports about its efficacy. Secondly, the cost of the device was affordable, and thirdly, the device was simple enough for the patient to use at home, avoiding repeated visits to the clinic. The facts that the device was completely safe, and that no adverse side effects had ever occurred, were also important considerations. In addition, the comblike teeth of the device separates the hair so that the nine laser beams emitted can easily reach the scalp without absorption or interference from existing hair, and the laser beams themselves are visible light energy allowing the patients to see for themselves that the device is operating. In total, 35 patients with androgenetic alopecia (AGA) were involved in the study, of which 28 were males, aged 28 to 72, and 7 were females, aged 46 to 76. Each patient was given a HairMax LaserComb to use at home for six full months and was instructed to comb his/her entire scalp hair slowly (advancing one-quarter of an inch per second) for five to ten minutes every other day. The treatment was to be carried out when the hair and scalp were clean and the hair was dry. A hair count in the greatest area of alopecia and a determination of the tensile strength of the hair was carried out before treatment was started and again after six months of treatment. As each patient was troubled by AGA to start with, the authors did not want to clip additional hair for the hair counts. Accordingly, they created a clear acrylic mold of each patient's head, with the front of the mold positioned at the hairline, and a one-centimeter square removed from the mold in the area of greatest alopecia in either the temporal or vertex region. For the hair count, the hairs within the one square centimeter space were pulled through the opening, and then counted using a surgical skin hook and a lens with five times magnification. Hair counts were carried out by both authors to confirm the accuracy of the data. To test the tensile strength of the hair before treatment and after six months of treatment, three typical terminal hairs were removed from the one square centimeter area and a VIP HairOScope (Belson Imports, Hialeah, Fla.) used to determine the tensile strength.

### Hair Regrowth and Increased Hair Tensile Strength in the Study

TABLE 1
HAIR MAXLASERCOMB HAIR COUNT DATA

| Hair Count | | | | | | | | |
|---|---|---|---|---|---|---|---|---|
| Number | Patient | Age | Sex | Area | Baseline | 6 months after | Difference | Change (%) |
| 1 | RC | 47 | M | T | 23 | 45 | 22 | 95.7 |
| 2 | CP | 34 | M | T | 33 | 51 | 18 | 54.5 |
| 3 | DL | 28 | M | T | 22 | 31 | 9 | 40.9 |
| 4 | SH | 56 | M | T | 6 | 11 | 5 | 83.3 |
| 5 | WG | 35 | M | T | 16 | 28 | 12 | 75.0 |
| 6 | JC | 29 | M | T | 38 | S6 | 18 | 47.4 |
| 7 | TM | 34 | M | T | 12 | 46 | 34 | 283.3 |
| 8 | JH | 51 | M | T | 18 | 27 | 9 | 50.0 |
| 9 | LM | 63 | M | T | 16 | 16 | 0 | 0.0 |
| 10 | Jf | 29 | M | T | 15 | 23 | 8 | 53.3 |
| 11 | AT | 36 | M | T | 28 | 37 | 9 | 32.1 |
| Average (T) | | 40.2 | | | 20.6 | 33.7 | 13.1 | 74.1 |
| 12 | DB | 55 | M | V | 8 | 22 | 14 | 175.0 |
| 13 | RK | 37 | M | V | 36 | 41 | 5 | 13.9 |
| 14 | PP | 29 | M | V | 22 | 61 | 39 | 177.3 |
| 15 | EL | 34 | M | V | 19 | 36 | 17 | 89.5 |
| 16 | JI | 51 | M | V | 18 | 27 | 9 | 50.0 |
| 17 | BG | 48 | M | V | 14 | 64 | 50 | 357.1 |
| 18 | SA | 59 | M | V | 12 | 23 | 11 | 91.7 |
| 19 | DB | 29 | M | V | 18 | 26 | 8 | 44.4 |
| 20 | EW | 38 | M | V | 22 | 28 | 6 | 27.3 |
| 21 | JS | 56 | M | V | 18 | 31 | 13 | 72.2 |
| 22 | MB | 35 | M | V | 22 | 39 | 17 | 77.3 |
| 23 | PL | 46 | M | V | 12 | 23 | 11 | 91.7 |
| 24 | JL | 72 | M | V | 12 | 33 | 21 | 175.0 |
| 25 | CR | 42 | M | V | 12 | 23 | 11 | 91.7 |
| 26 | PH | 60 | M | V | 12 | 27 | 15 | 125.0 |
| 27 | RH | 42 | M | V | 24 | 38 | 14 | 58.3 |
| 28 | JB | 30 | M | V | 4 | 17 | 13 | 325.0 |
| Average (V) | | 44.9 | | | 16.8 | 32.9 | 16.1 | 120.1 |
| Average (V 1 T) | | 43.1 | | | 18.2 | 33.2 | 15.0 | 102.7 |
| 29 | JL | 56 | F | T | 32 | 51 | 19 | 59.4 |
| 30 | FP | 66 | F | T | 19 | 27 | 8 | 42.1 |
| 31 | EL | 71 | F | T | 22 | 29 | 7 | 31.8 |
| 32 | LW | 46 | F | T | 8 | 15 | 7 | 87.5 |
| Average (T) | | 59.8 | | | 20.3 | 30.5 | 10.3 | 55.2 |
| 33 | NC | 64 | F | V | 19 | 29 | 10 | 52.6 |
| 34 | PJ | 76 | F | V | 18 | 36 | 18 | 100.0 |
| 35 | RM | 49 | F | V | 19 | 27 | 8 | 42.1 |

| | | | | | |
|---|---|---|---|---|---|
| Average (V) | 63.0 | 18.7 | 30.7 | 12.0 | 64.9 |
| Average (T 1 V) | 59.8 | 20.3 | 30.5 | 10.3 | 55.2 |
| Average (T, M 1 F) | 45.4 | 20.5 | 32.9 | 12.3 | 69.1 |
| Average (V. M 1 F) | 47.6 | 17.1 | 32.6 | 15.5 | 111.9 |
| Average (T 1 V, | 46.7 | 18.5 | 32.7 | 14.1 | 93.5 |
| M 1 F) | | | | | |

M, male;
F, female;
T, temporal;
V, vertex.

### Results of the Study

The scientific data for the hair counts is demonstrated in Table 1, and the data for the tensile strength of the hair recorded in Table 2. One-third of the patients did report some temporary slightly increased hair shedding during the first one or two months of treatment, but after two months, this no longer occurred. In summary, Table 1 shows that the hair counts increased in the temporal area an average of 55.2% in women, 74.1% in men, and 69.1% for all patients. In the vertex area, the corresponding percentages were 64.9% for women, 120.1% for men, and 111.9% for all patients. There was a hair count increase of 93.5% when all temporal and vertex patients were combined. In general, males and the vertex area did the best, but both sexes and all areas did demonstrate significant improvement.

### Satino and Markou

TABLE 2
HAIRMAXLASERCOMB HAIR TENSILE STRENGTH DATA

| Hair tensile | | | | | | | | |
|---|---|---|---|---|---|---|---|---|
| Number | Patient | Age | Sex | Area | Baseline | 6 months after | Difference | Change (%) |
| 1 | RC | 47 | M | T | 4.5 | 5.9 | 1.4 | 31.1 |
| 2 | CP | 34 | M | T | 3.8 | 6.1 | 2.3 | 60.5 |
| 3 | DL | 28 | M | T | 3.7 | 4.2 | 0.5 | 13.5 |
| 4 | SH | 56 | M | T | 2.1 | 3.5 | 1.4 | 66.7 |
| 5 | WG | 35 | M | T | 2.3 | 3.6 | 1.3 | 56.5 |
| 6 | JC | 29 | M | T | 2.4 | 4.1 | 1.7 | 70.8 |
| 7 | TM | 34 | M | T | 1.2 | 5.2 | 4.0 | 333.3 |
| 8 | JH | 51 | M | T | 5.5 | 6.3 | 0.8 | 14.5 |
| 9 | LM | 63 | M | T | 2.4 | 2.9 | 0.5 | 20.8 |
| 10 | JT | 29 | M | T | 4.2 | 4.9 | 0.7 | 16.7 |
| 11 | AT | 36 | M | T | 4.6 | 5.7 | 1.1 | 23.9 |
| Average (T) | | 40.2 | | | 3.3 | 4.8 | 1.4 | 64.4 |
| 12 | DB | 55 | M | V | 1.6 | 3.7 | 2.1 | 131.3 |
| 13 | RK | 37 | M | V | 4.0 | 5.8 | 1.8 | 45.0 |
| 14 | PP | 29 | M | V | 2.9 | 5.8 | 2.9 | 100.0 |
| 15 | EL | 34 | M | V | 4.1 | 5.4 | 1.3 | 31.7 |
| 16 | JI | 51 | M | V | 1.9 | 3.1 | 1.2 | 63.2 |
| 17 | BG | 48 | M | V | 3.5 | 4.7 | 1.2 | 34.3 |
| 18 | SA | 59 | M | V | 2.3 | 5.0 | 2.7 | 117.4 |
| 19 | DB | 29 | M | V | too short | | N/A | N/A |
| 20 | EW | 38 | M | V | 2.9 | 3.1 | 0.2 | 6.9 |
| 21 | JS | 56 | M | V | 1.1 | 3.1 | 2.0 | 181.8 |
| 22 | MB | 35 | M | V | 0.8 | 2.3 | 1.5 | 187.5 |
| 23 | PL | 46 | M | V | 1.7 | 4.0 | 2.3 | 135.3 |
| 24 | JL | 72 | M | V | 2.9 | 4.6 | 1.7 | 58.6 |
| 25 | CR | 42 | M | V | 2.8 | 4.3 | 1.5 | 53.6 |
| 26 | PH | 60 | M | V | 2.4 | 4.3 | 1.9 | 79.2 |
| 27 | RH | 42 | M | V | 1.3 | 3.5 | 2.2 | 169.2 |
| 28 | JB | 30 | M | V | 3.8 | 5.1 | 1.3 | 34.2 |
| Average (V) | | 44.9 | | | 2.5 | 4.2 | 1.7 | 89.3 |
| Average (V 1 T) | | 43.1 | | | 2.8 | 4.4 | 1.6 | 79.5 |
| 29 | JL | 56 | F | T | 2.0 | 4.6 | 2.6 | 130.0 |
| 30 | FP | 66 | F | T | 3.2 | 3.8 | 0.6 | 18.8 |
| 31 | EL | 71 | F | T | 2.2 | 4.0 | 1.80 | 81.8 |
| 32 | LW | 46 | F | T | 2.7 | 5.4 | 2.7 | 100.0 |
| Average (T) | | 59.8 | | | 2.5 | 4.5 | 1.9 | 82.6 |
| 33 | NC | 64 | F | V | 1.7 | 2.9 | 1.2 | 70.6 |
| 34 | PJ | 76 | F | V | 2.1 | 3.4 | 1.3 | 61.9 |
| 35 | RM | 49 | F | V | 2.1 | 3.8 | 1.7 | 81.0 |

| | | | | | |
|---|---|---|---|---|---|
| Average (V) | 63.0 | 2.0 | 3.4 | 1.4 | 71.1 |
| Average (T 1 V) | 59.8 | 2.5 | 4.5 | 1.9 | 82.6 |
| Average (T, M 1 F) | 45.4 | 3.1 | 4.7 | 1.6 | 69.3 |
| Average (V, M 1 F) | 47.6 | 2.4 | 4.1 | 1.7 | 86.4 |
| Average (T 1 V, | 46.7 | 2.7 | 4.4 | 1.6 | 78.9 |
| M 1 F) | | | | | |

M, male,
F, female,
T, temporal;
V, vertex.

Similarly, in Table 2, the hair tensile strength increased in the temporal area 82.6% in women, 64.4% in men, and 69.3% in both sexes. In the vertex area, the percentages were 71.1% for women, 89.3% for men, and 86.4% for both sexes. The hair tensile strength was increased 78.9% when all temporal and vertex patients were considered. There was greater improvement in the vertex area in males, but more improvement in the temporal area in females. Both sexes and all areas did benefit significantly.

### Discussion of the Study

In general, the results far exceeded the expectations of the authors, and they were pleased to be able to document the benefits that LLLT with the HairMax LaserComb can achieve for both men and women in both the temporal and vertex regions. Although there were four times as many men as women patients in the study, each sex did demonstrate significant benefits from the LLLT. The mechanism or mechanisms of action of LLLT are unknown with regard to the stimulation of hair growth or how the hair tensile strength is increased so greatly. From wound healing studies, it is known that LLLT causes an increase in the microcirculation of tissue and a reduction in inflammation.₍₂₎ The amount of cellular energy in the form of adenosine triphosphatase (ATP) is also increased following LLLT.₍₂₎ Perhaps one or more of these beneficial effects are responsible for the results that we were able to achieve. The authors hypothesized that the early temporary hair shedding experienced by some patients was most likely related to an accelerated hair cycle in general. Obviously, more research is required if we are to fully understand the scientific findings noted in this paper.

### Conclusion of the Study

LLLT with the HairMax LaserComb is an effective treatment for stimulating hair growth and increasing the tensile strength of hair in both sexes in both the temporal and vertex regions. In the authors' opinion, LLLT should be given serious consideration as an option in the treatment of AGA in view of its safety, ease of patient home administration, and the benefits documented in this study.

### REFERENCES OF THE STUDY

1. LaserWorld LLLTInternet Guide (http://www.laser.nu/).
2. Pontinen, P.: Low Level Laser Therapy as a Medical Treatment Modality. Art Urpo, Ltd., Publishers, 1992, pp 99-101.
3. Trelles, M, and Mayayo, E.: The Growth of Hair under Influence of the He—Ne Laser Beam: Histological Study. Sorrento. World Congress of Laser-Therapy, 1982.
4. Trelles, M, Mayayo, E., Schmidt, C., Igllesias, J, and Barber, J.: Laser Para la Salud y la Estetica. Etecnes, 2nd. edition, 1983, pp 98-107.
5. Unger, M: Low Level Laser Therapy (LLLT) for Hair Biostimulation, 9th Annual Meeting of the International Society of Hair Restoration Surgery, Puerto Vallarta,

### SUMMARY OF THE INVENTION

The present invention and system in one embodiment, is a portable, wearable therapeutic hair, scalp, skin, and hearing treatment light emitting device, system, and method for: reducing hair loss in men and women; treating loss of hair color; reducing and eliminating dandruff; increasing hair tensile strength; stimulating new hair growth; and reversing hearing loss in certain cases, and eliminating Tinnitus, and Vertigo by supplying laser light and/or light from a Light Emitting Diode (LED) to a user's head and/or ears by means of a hat, which can, but does not necessarily have to be used in conjunction with a special topical Minoxidil and Finasteride mixture to be applied to the scalp, twice a day in the case of male pattern baldness, and a Ketoconazole shampoo to wash the hair with. The device also aids in the therapeutic treatment of eczema, psoriasis, gum recession, wrinkles, burns and skin wounds, some skin cancers, acne, enlarged pores, oily skin, stretch marks, Folliculitis, Arthritis, back pain, Bursitis, Carpal Tunnel Syndrome, headaches, neck pain, repetitive stress injuries, TMJ pain, tendonitis, sprains, swelling, excessive perspiration, viral warts, precancerous solar keratosis, sun damaged skin, skin ulcers, cellulite, damaged DNA due to environmental exposure to the sun, sunburn, insomnia, jet lag, immune dysfunction disorders, aging, scar tissue, and stimulates collagen. The device is used by the individual and does not require trained personnel to provide a treatment.

The device provides an on-off switch for stationary, pulsating, and/or oscillating laser light and/or LED light to the scalp while wearing a hat-like device, making the device portable anywhere. The electronics can be removed as one unit to be installed in other styles of hats, making it extremely versatile. In one embodiment, a unique, stepped, beam-splitting reflector splits each single laser beam to allow an individual laser to simultaneously provide multiple laser beams, which are distributed across a segment of an individual's scalp. The reflector is mechanically aligned with the laser source and has a zigzag structure, which mechanically deflects portions of the beam as it passes over the peaks of the reflector. The portions of the laser beam form a line of laser beams that project toward the user's scalp. Multiple parallel rows of such laser beams are utilized thereby encompassing the entire scalp with the light. The individual lasers can uniquely snap or screw into place for easily changing laser wavelengths and adding attachments in the treatment of other skin disorders.

The device includes a current generator (removable batteries attached to the inside housing of the device, or solar panels, or external power such as AC plug or car plug) disposed within a housing, configured to output a current for passage into laser sources and/or other LED's also disposed within the housing. Any style hat can be utilized. A solar cell(s) can be mounted to the top exterior of the hat, using daylight to produce an electric current that can run the device, or maintain the batteries' charge, or recharge them if necessary. The output and direct respective portions of the laser beams radiate inward from the hair and scalp treatment device toward the user's head when the hair and scalp treatment device is in use. The interior of the hat is a mirrored reflective silvery surface, such as chrome, and has an adjustable headband to fit any head size. The method comprises the step of directing a series of different laser and/or LED treatments to the thinning portions of the scalp, and/or flaps with lasers that descend over the ears (in the case of hearing loss), and/or attachments for treating receding gums in a series of pulsating, oscillating, and/or stationary laser/LED light beams. The device also contains the capability of maximizing battery power by switching off specific lasers or LED's as the battery (ies) begins to lose power. Each row of lasers may contain an on-off switch so that as the batteries begin to expire, individual rows may be turned off to maximize remaining battery power. The preferred embodiment of the device also contains a manual switch affixed to the housing which turns the device on-off, and switches between stationary, pulsating, or oscillating features, thereby maximizing battery power. This device also contains a separate manual switch affixed to the housing to allow for easy change between solar power, battery power, Alternate Current, or Car Adapter. The unique individual lasers can snap or screw into place for easily changing laser wavelengths in the treatment of various different skin disorders. The hair/hearing restoration device is comprised of a lightweight hat body, housing a plurality of wide pattern laser diodes configured to cooperate in projecting a precision laser light pattern at focal planes proximate to the skin area to be irradiated. The lasers may also be adjusted by the user in the case of hair loss, to focus them on the areas of the scalp that require treatment, since every case of hair loss is unique to the individual. The device slips into a mirrored enclosed base containing a flat piece of clear glass atop it for use as a reflective surface for ease in treating other skin disorders found on other parts of the body. The laser device can be used alone in both men and women, or treatment for hair loss in men can be combined with the unique topical Minoxidil-Finasteride mixture, and Ketoconazole shampoo for faster results. The preferred dosage is, but not limited to: the 2 oz. topical mixture containing 5% Minoxidil with 25 mg. of Finasteride in a solution, suspension, emulsion, or any other mixture type, to be applied topically to the scalp at a rate of, but not limited to, 1 ml. per application, twice daily, and 2% Ketoconazole shampoo to wash the hair with. At this point in time, a prescription probably would be needed from a doctor for the topical solution.

The portable body treatment device contains a portable “chamber” that one can sit in, and uses all the same principles as the head treatment device except on a larger scale, and for treating various skin disorders on the rest of the body. The unique snap in or screw in lasers or LED's can be easily changed to alter the desired wavelength in order to treat a plethora of skin disorders. The chamber folds for easy storage and transport.

### DETAILED DESCRIPTION OF THE DRAWINGS

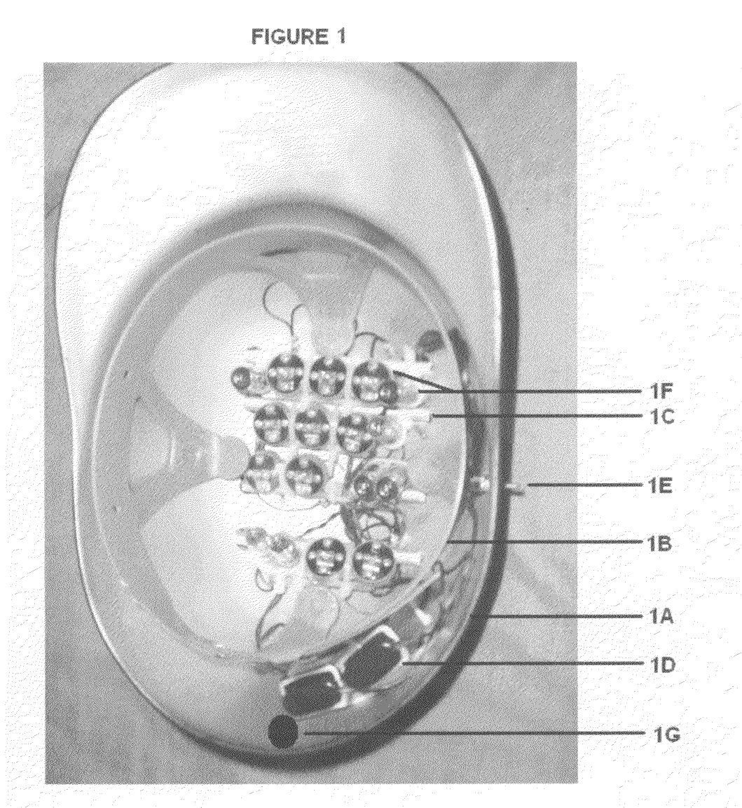

FIG. 1 is a view of the underside of a preferred embodiment of the invention, which illustrates 4 parallel rows of lasers extending from right to left through the interior of the device, the batteries, and the on-off switch.

FIG. 1 (1A) is the bottom view of the housing of a preferred embodiment of the device which shows the device is in the form of a hat or helmet composed of lightweight plastic, fiberglass, fabric material, light metal, graphite composite, or any suitable lightweight material.

FIG. 1 (1B) is a bottom view of a preferred embodiment of the device which illustrates the one-size fits all plastic headband that is designed to rest high up on the head, and is fully adjustable for all head sizes.

FIG. 1 (1C) is a bottom view of a preferred embodiment of the device, which illustrates the tracks, which are affixed to the interior of the hat. The tracks can be moveable based on the individual's needs. The tracks hold the lasers and can rotate on their axis to oscillate the lasers.

FIG. 1 (1D) is a bottom view of a preferred embodiment of the device in which the batteries are shown housing 9-volt batteries. Number of batteries can vary depending on strength of lasers used, and number of lasers used.

FIG. 1 (1E) is a bottom view of a preferred embodiment of the device displaying the ON-OFF switch mounted on the side of the exterior of the hat. Positions can include Off, Stationary, Pulsating, and Oscillating settings.

FIG. 1 (1F) is a bottom view of a preferred embodiment of the individual Class IIIA laser diodes with a multi-wavelength range most effective from 575 nanometers to 1600 nanometers, for growing hair, but the lasers/LED's can be easily changed to a different wavelength, for treating different problems.

FIG. 1 (1G) is a bottom view of a preferred embodiment of the device displaying the connector to the base unit of the device.

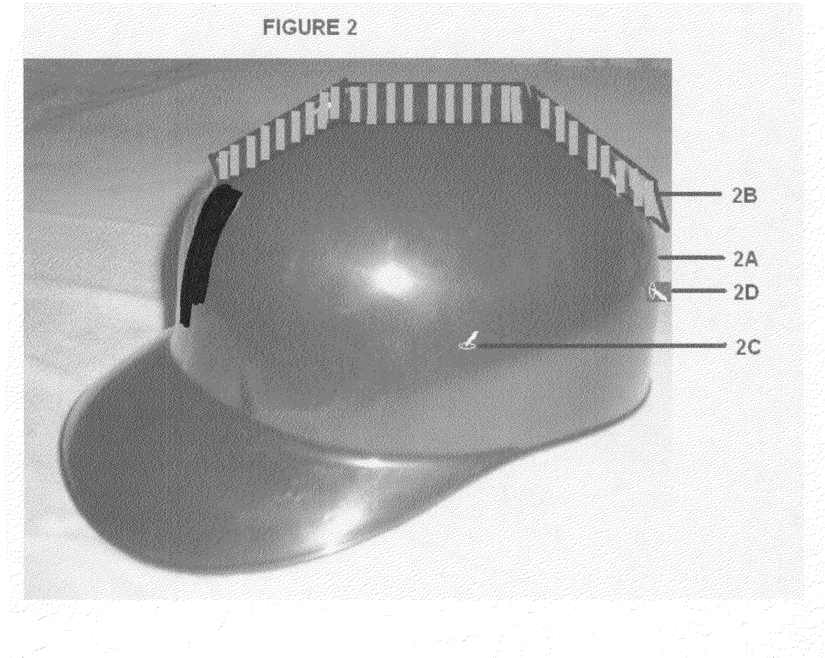

FIG. 2 is a side view of a preferred embodiment of the exterior of the helmet. Appears like a normal baseball hat/helmet, but can be made more stylish like other types of hats.

FIG. 2 (2A) is an illustration of a preferred embodiment of the exterior of the hat.

FIG. 2 (2B) is a side view of a preferred embodiment of the device, which illustrates the solar panels on the top of the device.

FIG. 2 (2C) is a side view of a preferred embodiment of the device, which illustrates the on off pulsate-oscillate switch on the side of the device.

FIG. 2 (2D) is a side view of a preferred embodiment of the device which illustrates the solar-battery-Alternate Current switch on the rear of the device.

FIG. 2 (2E) is a side view of a preferred embodiment of the device that illustrates the hinge and/or snap of the hearing restoration adapter on the side of the device.

FIG. 2 (2F) is a side view of a preferred embodiment of the device, which illustrates the removable flaps of the hearing restoration adapter on both sides of the device.

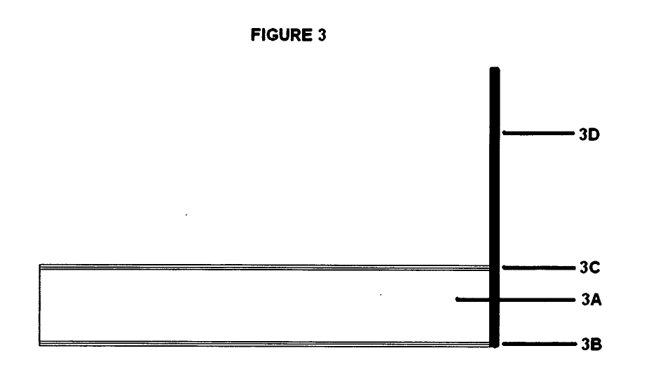

FIG. 3 is a side view of a preferred embodiment of the base and attachment for the helmet, in the treatment of other parts of the body.

FIG. 3 (3A) is a side view of a preferred embodiment of the hollow box of the base.

FIG. 3 (3B) is a side view of a preferred embodiment of the mirrored bottom of the box of the base.

FIG. 3 (3C) is a side view of a preferred embodiment of the clear glass resting on top of the box of the base.

FIG. 3 (3D) is a side view of a preferred embodiment of the support between the box base and the hat device.

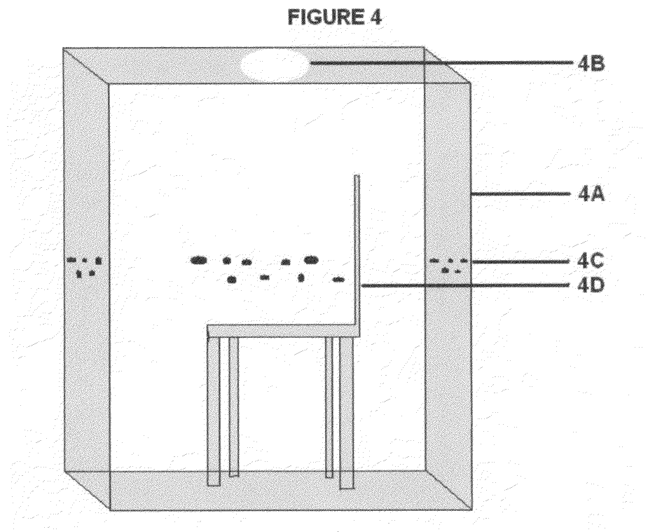

FIG. 4 is a side view of a preferred embodiment of the body “chamber” treatment device.

FIG. 4 (4A) is a side view of a preferred embodiment of the lightweight, foldable box, large enough for treatment of a human body, with a completely mirrored interior.

FIG. 4 (4B) is a side view of a preferred embodiment of the hole in the lid, for the individual's neck to fit through.

FIG. 4 (4C) is a side view of the preferred embodiment of the laser modules/LED's in the walls of the chamber.

FIG. 4 (4D) is a side view of a clear chair whereby someone can sit comfortably inside the chamber.

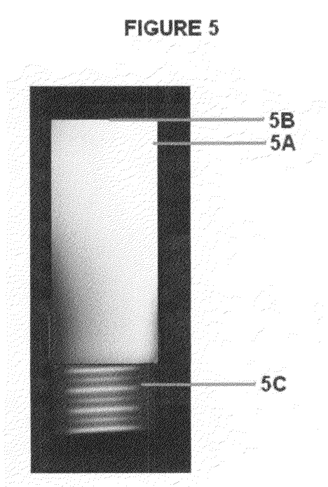

FIG. 5 is a side view of the preferred embodiment of the screw in laser module.

FIG. 5 (5A) is a side view of the easily removable laser module.

FIG. 5 (5B) is a side view of the aperture.

FIG. 5 (5C) is a side view of the screw in device at the base of the laser module, whereby one can easily change modules.

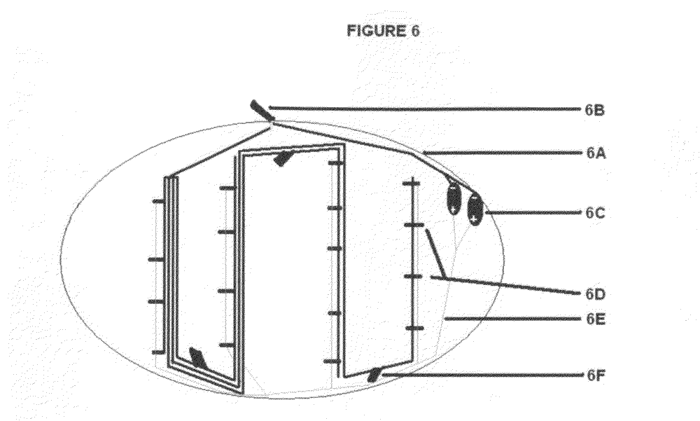

FIG. 6 is a bottom view of the electronics of the head device.

FIG. 6 (6A) is a bottom view of the hat housing.

FIG. 6 (6B) is a bottom view of the main on-off switch (which may contain an option for pulsating, oscillating, and/or stationary beams.)

FIG. 6 (6C) is a bottom view of the batteries powering the device.

FIG. 6 (6D) are the laser modules.

FIG. 6 (6E) is the positive wire connecting all the lasers and/or LED's to the batteries.

FIG. 6 (6F) are optional automatic/manual switches for each separate row, whereby as the power fades, the unit turns off one row at a time to maximize the power remaining.

### DETAILED DESCRIPTION OF THE PREFERRED EMBODIMENT

Prior to a detailed discussion of the figures, a general overview of the system will be presented. For ease of discussion, the term “scalp” will be used to describe the conventional definition which describes the hair covered portion of skin on the user's head, and in addition, any skin surface where hair is desired to be grown (e.g., the beard, etc). The following detailed description is of the best presently contemplated mode of carrying out the invention. This description is not to be taken in a limiting sense, but is made merely for the purpose of illustrating the general principles of the invention. The scope of the invention is best defined by the appended claims.

In a preferred embodiment, the present invention is directed to a process for dermatological treatment. Such a treatment may include the photostimulation of hair follicles, hair bulb, hair bulge, stem cells and the surrounding tissue to produce temporary or permanent stimulation of activity in the surrounding tissue or supporting tissue in human or mammalian skin, of some or all of the hairs. In a preferred embodiment the process produces little or no permanent injury or damage to nearby skin tissue. Primarily, only the hair and immediately surrounding tissue are affected. For purposes of the present invention, any recitation of the hair also includes the hair follicle, bulb, bulge, stem cells and other components of the supporting dermal structure that supports hair growth. In a process according to one embodiment of the present invention, an agent may be selected which is capable of penetrating the hair ducts and attaching; bonding or otherwise becoming incorporated into the hair shaft, hair follicle, hair bulb, hair duct cells, or stem cells collectively referred to hereinafter as hair growth structures. The agent may be characterized as an active agent in that it performs a function in addition to simply occupying or contaminating the space in the ducts surrounding the hair shaft. The agent may have sufficient optical absorption of a wavelength (or a combination of wavelengths) of a coherent or non-coherent light source which can penetrate the skin adequately to be absorbed by the target agent or the new agent-tissue complex or it may in some other way directly or indirectly enhance the stimulation of hair growth structures. The area of skin overlying where the hair duct is located may be cleansed. After the skin is cleansed, the skin may be treated to improve permeability. This may be accomplished, for example, by treating the skin with steam or a hot moist towel to hydrate the skin and hair or removing a portion of the stratum corneum through various means known in the art, exemplary of which is microdermabrasion. The agent may be applied in sufficient quantity and in suitable form to be incorporated into the target tissue in adequate or optimal amounts to allow the production of the desired tissue effect. Excess agent may be removed, neutralized, inactivated, decolorized, diluted or otherwise altered so that residual contamination of the skin by such excess agent is either (a) absent and does not interact with the light or energy source, or (b) present in such small quantity that it provides no clinical effect.

Delivery of the desired agent into the target tissues, ducts, or nearby sebaceous oil glands may be enhanced, facilitated or made possible by the use of enzymes capable of altering the structure, permeability, or other physical characteristics of the stratum corneum or by the use of ultrasound or phonophoresis either for penetration into the gland or surrounding target tissues or, once penetrated, to cause the release of the agent from the encapsulated delivery device such as liposomes, polymers, microspheres, etc. so as to cause penetration or attachment of this active agent. Ultrasound may be used therapeutically to interact directly with the agent or the agent-tissue complex to produce the desired damaged target tissues (to be used alone or in combination with laser or non-laser light sources).

Microdermabrasion may also be used to permit greater penetration of the skin, wherein the upper epithelial layers are removed. These layers create a natural barrier to the permeability of the skin and by their removal, penetration of the skin by topical agents is facilitated. This method may be further enhanced by using ultrasound, alone or in combination with alteration of the stratum corneum, to further improve the performance of topical compositions. Although preferred embodiments of the present invention may use LEDs, ultrasound, and/or laser or light energy from sources such as light-emitting diodes, the present invention is not limited to the use of these energy sources. Other sources of energy, including (without limitation) microwave energy and radio frequency energy or electrical stimulation or magnetic fields/forces may also be used. Exemplary of known light sources are fluorescent lights, flashlamps, filamentous lights, metal halide lights, halogen lights, etc. One skilled in the art will recognize that any light source capable of emitting electromagnetic radiation at a medically useful wavelength, as described herein, directly, or by means of optical filtration, is within the scope of suitable light sources according to the present invention. For purposes of the photomodulatory and photothermal treatment methods described, any source capable of emitting light having a wavelength from about 300 nm to about 1600 nm, or producing electromagnetic radiation which is filtered or otherwise altered to exposure the skin, a topical composition, or other component of the present treatment regime to a wavelength of light in the aforementioned range is medically useful. The targeted skin may be exposed to one or more wavelengths of LED, laser or non-laser light such as filtered filamentous sources or fluorescent sources alone or in combination with single or multiple frequencies of ultrasound. The light source may be polarized or unpolarized, as can any light source described herein in accordance with the present invention. A variety of parameters may be used (including pulse duration, energy, single or multiple pulses, the interval between pulses, the total number of pulses, etc.) to deliver sufficient cumulative energy to interact with the agent or tissue complex. In one embodiment of the invention, this results in the stimulation of hair growth or the supporting skin tissue through photomodulatory means, photothermal means electrical stimulation, or combinations thereof. Alternatively, proper exposure to certain wavelengths of light, combinations of certain wavelengths of light, such light sources either alone in combination at various intensity levels, with and without topical compositions to enhance the penetration of the light, are capable of photostimulation of hair follicles, glandular and duct activity, etc. resulting in the stimulation of hair growth. Ultrasound may also be used to preheat the target structures or the entire skin. Further for treatment over a broad area of human skin, the light source may be diffused through a device such as a holographic diffuser; or, alternatively, the light source may be comprised of an array of individual emitters such as the multi-panel array of LEDs. Further increasing the number of panels to more precisely follow the contours of the portion of the patient receiving treatment produces more uniform exposure and improved results. For example, a collection of panels that can be manipulated to provide even exposure to the entire scalp of a patient will provide excellent results when the scalp area is targeted for treatment to stimulate hair growth. Since LED sources are generally considered “insignificant risk devices,” no medical supervision is required and these devices may be used by the patient for at-home treatment or as part of an ongoing skin-care system after receiving treatment by a physician.

It has been found that the application of low-power lasers can stimulate natural processes within the body, and particularly, within the scalp such that a variety of therapeutic results can be achieved. There has been, for example, a substantial amount of research done on the use of laser energy to stimulate the body's natural processes to reverse hearing loss, heal wounds, apply skin therapy, etc. The advantage of using the coherent light of a laser, is that laser light tends to concentrate power such that a greater effect can result from the application of a particular energy level. However, LED light will also produce therapeutic and healing results when applied to the skin. It only differs from laser light in the sense that its energy is not concentrated in the form of a laser beam. The device described herein has several embodiments including a laser embodiment which emits only coherent light, a non-laser embodiment which emits only non-coherent light, and a mixed embodiment which emits both coherent and non-coherent light. The general discussion of the laser, the non-laser, and the next embodiments will now be presented.

It has been found that low-power lasers may be used in a variety of therapeutic applications. For example, low-power lasers are used widely for a variety of cosmetic applications such as skin care, scar reduction, wound healing, as well as reversal of hearing loss and the like. In addition, it has also been found that the application of low-power laser light to an individual's scalp will assist the hair's natural ongoing replacement process and improve the scalp's condition.

One of several factors associated with the use of lasers to stimulate hair growth is that laser treatments tend to increase scalp blood circulation. In fact, studies have shown that the application of laser energy to the scalp of a user can increase scalp blood circulation by more than fifty percent without significant changes in scalp temperature. This results in the skin receiving a more abundant supply of nutrients, and in turn, the structures in the skin, such as hair follicles, also receive a more abundant supply of nutrients and necessary materials from the body.

Microscopic studies have shown that laser energy increases ATP energy (Adenosine Triphosphate) levels in the cells; circulation and oxygenation of the blood to the scalp and hair bulb; removes calcification and blockages around the hair bulb; as well as increases cell replacement or regenerative activity. These factors help hair to improve in fullness, shine, body and elasticity. Problems such as over-oily or dry scalp, dandruff and itchiness can also be reduced. Research on the use of cold beam lasers indicates that application of a cold beam laser to an individual's scalp will normalize metabolism of tissues, improve tropism (blood cell nutrition), and assure a regular sebaceous secretion. Measurements taken from scalps treated by cold laser indicate that hair bulbs are strengthened, hair growth can be measurably ascertained, and hair color will darken.

The increase in blood flow helps as follows: in the human scalp, the follicle in which the hair grows is attached to the scalp by a structure known as the Papilla. The Papilla provides a path for nutrients in the blood to reach the cells in the hair. The laser treatment described herein improves both the condition of the Papilla itself, as well as the blood flow reaching the Papilla. The unique structure of the wearable device presented herein provides a path for laser light, and/or LED light, to the Papilla, which results in the scalp being bathed in laser light energy.

In the case of hearing loss, Tinnitus, and Vertigo, the inner ear contains two sensory organs: the organ of balance in the Labyrinth, and the organ of Corti (hearing) in the Cochlea. In the Cochlea, the waves of vibration (sound) mechanically transmitted there via the Eardrum and the Ossicles (middle ear) are first compressed by means of a highly complex fluid system and then converted into electro-physiologic impulses that are passed on to the central nervous system. This conversion process can be explained in detail with today's biological knowledge. It takes place in the auditory cells of the Cochlea and is connected with movements of hair-like receptors on the cellular membrane and other processes within the cells of the inner ear. For this to happen properly the auditory cell requires energy in the form of ATP. ATP is produced by the mitochondria (cellular power plants) inside each cell.

If the Cochlea is acutely or chronically over strained, its sensory (auditory) cells and their various cellular organs are affected as well, and they inevitably lose part of their functional capacity. The cells suffer from a lack of ATP. A continuous lack of ATP within the inner ear cells of the Cochlea leads to either a gradual or sudden impairment of the entire hearing organ. The patient experiences this as acute or insidious symptoms such as: pressure in the ear, hearing loss, or tinnitus (insidious or acute).

In the Semicircular Canals of the organ of balance, any changes in the position of our head and body are also conveyed via a fluid system and the movements of hair-like receptors. For this reason the ATP-consuming processes within the cells in question are almost identical to those in the Cochlea during the act of hearing: mechanically transmitted motions are first converted into electro-physiologic impulses and then passed on to the central nervous system. A continuous lack of ATP within the inner ear cells of the Labyrinth leads to either a gradual or sudden impairment of the entire organ of balance. The patient experiences this as acute or insidious symptoms such as pressure in the ear, and Vertigo (acute or insidious).

In addition to their similar functional properties, the Labyrinth and the Cochlea are connected by means of a common fluid system. This is why inner ear diseases often affect both organs at the same time and show a large variety of symptoms.

The extent of the respective impairment varies from person to person, but although these factors result in individually different healing processes, the low-level laser therapy is able to produce a positive biological reaction regardless of the dysfunction involved. This is due to its elementary and biologically compelling working mechanism.

On account of the basic conception of our cellular energy-converting system (collector principle), the electromagnetic energy released by the oxidation of nutrients is utilized as a source of primary energy for the production of the cellular fuel ATP. The antennae of the mitochondria can, in addition to the absorption of the released metabolic energy, utilize both the photons of the natural solar radiation (apparent bio-stimulative effect of sunlight on human cells) and the photons of low-level laser light (clinically proven bio-stimulative effect of low level laser light on human cells) as a source of primary energy.

The low-level laser therapy for inner ear dysfunction has been made use of, in Germany since the end of the eighties (for example by Dr. Uwe Witt, Hamburg and Dr. Lutz Wilden in Bad Fuessing) and is also to be found in quite a few other countries (Italy, France, Scandinavia, Switzerland, Hungary, Japan, Russia etc.) As the observation of healing processes has shown in the course of the years, even serious impairments such as the most excruciating cases of Tinnitus, hearing loss, Vertigo, and Meniere's Disease can be considerably alleviated and even healed with the aid of modern Low Level Laser Therapy (LLLT).

Each cell contains a number of power plants, called mitochondria. The function of these power plants is to produce ATP, the form of energy, which can be used by the cell to function properly. The inner mitochondrial membrane is a large collector surface folded onto a small total volume. All nutritive molecules (fats, proteins, and glucose) are oxidized within the cell to the last molecular nutritive component, that is, pyruvate. The pyruvate is then imported into the cellular power plant, where it disintegrates into carbon dioxide and water in the immediate proximity of the collector surface of the mitochondrion. The molecular bond energy thus released, which corresponds with visible ultraviolet and infrared light as far as its wavelength and frequency is concerned, is utilized by the mitochondrion to produce the cellular fuel ATP.

The collector surface of our cellular power plant is enlarged by the mushroom-shaped protrusions of the inner mitochondrial membrane. The molecular bond energy of the pyruvate is released in the form of light energy. The antennae pigments are capable of absorbing this light energy and transmit it to our cellular power plant, which can now produce the cellular fuel ATP. The natural solar radiation also stimulates the antennae pigments of the mitochondrion to produce ATP.

Low-level laser light is compressed light from the red and infrared spectrum of the electromagnetic radiation. It reaches the cellular power plants of low-lying cells as well and is absorbed by their collector surfaces as an additional source of energy. The cellular power plants can thus produce more ATP. The cellular energy is the fuel the inner ear cells strives after and needs. A sufficiently high supply of cellular energy enables our inner ear cell to work under optimum conditions and is the essential prerequisite to ensure a successful self-healing process. Low-level laser light increases the energy output in our cellular power plant.

A factor associated with the use of lasers is “energization.” Energization can be explained as follows: Light is energy. The use of a laser light on scalp and hair follicles or in the ears provides high levels of light, which are used by the cells in the scalp and hair, or hearing process to assist in the normal chemical processes performed by those cells. The scientifically agreed-upon term for this is photobiostimulation. The most common example of light converting into chemical energy is photosynthesis, where plants are fed via light converted into chemical energy. In a similar way, laser light penetrates into soft tissue and increases the action of Adenosine Triphosphate (ATP), a molecule that is a major carrier of energy from one reaction site to another in all living cells. By doing so, laser light increases the energy available to cells so they take in nutrients faster and get rid of waste products. Because of this benefit, scientists and physicians have been using low-level lasers over the past 30 years to accelerate wound healing and regenerate tissue.

Yet another factor associated with the use of laser light is known as “vibration.” Soft tissue and fluids in our bodies actually vibrate. The vibration occurs within a frequency range similar to that of cold-beam, red light laser. In fact, one scientific theory holds that cells are largely dependant for healthy function on an exchange of energy and information with surrounding cells. This is achieved via individual wave systems by which cells communicate through inter-connective plasma by vibration. A cell is in an unhealthy state when its vibrations become irregular or out-of-step with this common communications system. However, it can be brought back into vibratory “harmony” by being irradiated with low level lasers working at quantum level.

While the benefits of low-power laser treatments are known, attempts to take advantage of laser technology for the purpose of stimulating hair growth has produced limited results. In particular, the laser combs that are out on the market only use one or two low power lasers, reducing the effectiveness of the laser treatments. The device described herein, uses multiple low power lasers throughout the hat/helmet, thereby covering the entire scalp from different angles, and the laser light beams of the line generators are positioned parallel to each other, thereby covering the entire thinning scalp area. In one embodiment, combining this with LED light provides a broader spectrum of light to the scalp, thereby maximizing stimulation of the skin tissue.

Another problem related to prior art laser treatment devices is that they tend to be large devices which are heavy and immobile. As a result, they would usually be found in a salon or clinic where the user would be charged each time the user obtained a treatment. Further, since these devices typically have fixed locations, they would not be available to the user when traveling, and they would be inconvenient to access even when the user was not traveling.

The invention provided herein solves all of the foregoing problems. The invention is a lightweight wearable device which emits rows of laser beams which are produced by many laser beam generators, and each can then be split into multiple laser beams by a zig-zag shaped reflector. The hat provides more laser energy directly to the scalp and/or ears of the individual, and can be worn for long periods of time while doing other activities. In one embodiment, the ability to provide multiple beams from each single laser source is provided by a unique “zig-zag” lens which splits a single laser beam generated from a single laser device into multiple parallel beams. Each beam has a power level substantially similar to adjacent beams due to their generation by each single laser source.

It has also been found that stimulation of the skin using LED light also produces beneficial results. In particular, LED light will stimulate the skin such that the ability of the skin to nourish and produce thick healthy hair is enhanced in the same manner as was done by the laser. As was the case above, the hat structure with several light emitting modules used by the laser based embodiment will effectively allow access to substantial areas of the scalp by the LED light. It has also been found that a laser can be combined with an LED light source. This provides an advantage in that while the laser uses a narrow frequency bandwidth, the LED light source will deliver additional energy over a wider bandwidth. This maximizes the stimulation of the skin tissue and enhances its ability to support and nourish the growth of thick and healthy hair. In one embodiment, both laser energy and LED light energy can be selectively and independently activated to provide a user with the ability to use either or both energy sources.

The preferred embodiment of the device also contains a manual switch affixed to the housing which turns the device on-off, and switches between stationary, pulsating, and/or oscillating features, thereby maximizing battery power. The device also has an adapter which screws or snaps into one of the laser sockets, snaps into the sides of the hat, thereby hanging downward, and which can be positioned to shine laser light into both ears simultaneously, for the cases of restoring hearing loss, reversing Tinnitus, and eliminating Vertigo. This device also contains a separate manual switch affixed to the housing to allow for easy change between solar power, battery power, Alternate Current, or Car Adapter.

In the preferred embodiment of the device, each row of lasers may contain an automatic or manual on-off switch so that as the batteries begin to expire, individual rows may be turned off to maximize remaining battery power entering the remaining lasers. This would allow maximum draining of the battery power before each recharging session, thereby helping the batteries last longer, to maximize power. The preferred embodiment contains approximately, but not limited to, 9 Class IIIA, 5 mW lasers for every 9 volt battery used, so in the case of 18 lasers, two 9-volt batteries would be needed, but if stronger lasers are used, less lasers are needed. In the preferred embodiment, the lasers contain line, or elliptical generators, but can include any shape. The line generators are spaced parallel with each other for maximum coverage. The device slips into a base containing a mirrored interior, and a flat piece of clear glass above the base for use as a reflective surface in treating other skin disorders found on other parts of the body.

In the case of men and women, the laser device alone can be used for hair loss, or in males, treatment can be combined with the topical Minoxidil-Finasteride mixture for faster results. The preferred embodiment of the mixture is, but not limited to, a 2 oz. topical mixture, containing 5% Minoxidil with 25 mg. of Finasteride in a solution, suspension, or emulsion, or combination thereof, to be applied topically to the scalp at a rate of 1 ml. per application, twice daily, and Ketoconazole shampoo to wash the hair with. It is understood that a prescription might be needed for the topical Finasteride.

The preferred embodiment of the “chamber” device uses the same principles as the head device except that one's entire body can be treated at once in this portable, foldable, chamber, containing a clear chair for the individual to sit in, and numerous adjustable line, elliptical, and/or other shape producing interchangeable laser and/or LED modules. These modules can uniquely snap or screw into place in the reflective walls of the chamber. It contains all the same features as the head treatment device, except on a larger scale, and can therefore, in the case of skin disorders, treat an entire human body all at once in the privacy of one's own home. The top flaps of the chamber rest in place around the individual's neck, to prevent any stray light beams from entering one's eyes.

It has been found that lasers in other wavelengths, namely in the blue spectrum, can be used successfully to treat other types of skin disorders as is covered in the claims section. In the preferred embodiment of the device, the lasers can uniquely snap or screw into place, easily being changed from one wavelength to another to aid in the therapeutic healing of eczema, psoriasis, hearing loss, gum recession, wrinkles, burns and skin wounds, some skin cancers; treating acne, enlarged pores, oily skin, stretch marks, folliculitis, scars, excessive perspiration, viral warts, precancerous solar keratosis, sun damaged skin, skin ulcers, cellulite, damaged DNA due to environmental exposure to the sun, sunburn, insomnia, jet lag, immune dysfunction disorders, aging, and stimulating collagen as well as in the high red range for treating Arthritis, back pain, Bursitis, Carpal Tunnel Syndrome, headaches, neck pain, repetitive stress injuries, TMJ pain, tendonitis, sprains, and swelling.

While the invention has been described with respect to a preferred embodiment thereof, it will be understood by those skilled in the art that various changes in detail may be made therein without departing from the spirit, scope, and teaching of the invention. For example, the material used to construct the device may be anything suitable, the size and shape of the device can vary, the type of lasers and LED's can vary, etc. Accordingly, the invention herein disclosed is to be limited only as specified in the following claims.

I claim:

1. A portable, wearable hair, scalp, skin treatment, and hearing treatment device and system, comprising:
an easily changeable laser assembly, or many laser assemblies, and/or Light Emitting Diodes (LED's), and/or ultrasound, further comprising:
laser beam generator(s) and/or LED generator(s);
a power supply for supplying power to the laser beam generator(s) which may include but are not limited to:
batteries, solar cells, Alternate Current, car adaptor or other power sources; and
means to direct at least one laser beam and/or LED toward the scalp from the laser assembly; and
a screw-in, snap-in, or stick on (Velcro/tape), or any adhesive, laser adapter that can be inserted into one or both ears for the treatment of hearing loss; and
a screw in, snap in, or stick on (Velcro/tape), or any adhesive, laser adapter that has a flexible goose-neck, which can be positioned to shine onto the gums; and
a Minoxidil and Finasteride topical mixture, such as a solution, suspension, or emulsion, alone or in combination or in other forms common or known to those skilled in the art of topical and oral delivery to be applied to the scalp; and
a topical Ketoconazole shampoo, solution, suspension, or emulsion, alone, or in combination, or in other forms common or known to those skilled in art of topical and oral delivery to be applied to the scalp during the hair washing process, and to be used in conjunction with the other system parts mentioned;
whereby the scalp, gums, and/or ears of the individual are exposed during application of energy from the laser beam/LED to the scalp, gums, and/or ears, and hair growth may be expedited on the scalp due to the special topical solution, suspension, and/or emulsion, and shampoo.

2. A device as in claim 1, wherein:
each unique removable laser beam assembly and/or LED can snap or screw into place, which may further include:
a way of producing a plurality of pulsating, and/or oscillating, and/or stationary laser beam lines, ellipses, or any other shapes; each laser beam line, ellipse, or shape, spaced apart from one another and parallel or overlapping to irradiate the entire scalp area, and/or middle and inner ears, and/or gums; and
the entire electrical assembly can be removed in one piece and placed as a unit into other styles of hats by means of an adhesive such as, but not limited to, Velcro, snaps, etc;
whereby the amount of laser energy reaching the individual's scalp/ears/gums is maximized by the creation of many light beams.

3. A device, as in claim 2, wherein:
each laser generator and/or LED produces a single pulsating, and/or oscillating, and/or stationary laser beam line, ellipse, or other shape; and
a beam splitter may be used to split the single laser beam lines into a plurality of separate parallel laser beam lines;
whereby the device further produces multiple laser beams from each single laser beam generator.

4. A device, as in claim 3, wherein:
the beam splitter, is a beam reflector having a zigzag edge having a plurality of reflector teeth, each reflector tooth in the zigzag edge each reflecting a portion of the laser beam;
whereby the portions of the laser beam created by the reflector teeth each form an independent laser beam.

5. A wearable hair, scalp, skin treatment, and hearing treatment device as in claim 1, comprising:
a changeable laser assembly(ies) and/or LED, each further comprising:
an oscillating, and/or pulsating, and/or stationary laser beam generator which generates a laser beam line, and/or ellipse, and/or other shape; and
may include a beam splitter for splitting the laser beams into multiple substantially parallel laser beams and to direct the laser beams inward toward the scalp from the laser assemblies; and
a power source for supplying power to the laser beam generator including but not limited to:
rechargeable batteries housed within the apparatus, and/or solar cells attached to the outside top of the hat housing, with a connection for an alternate current plug and/or a car plug adapter;
whereby the scalp of the individual is exposed during application of energy from the laser beams and/or LED's to the scalp.

6. A device, as in claim 5, wherein:
the approximate output wavelength of a red-light pulsating and/or oscillating and/or stationary laser and/or LED is in the range of, but not limited to 575 to 1600 nanometers, for hair restoration and hearing loss, and the treatment of certain skin disorders such as reducing and eliminating dandruff, eczema, gum recession, psoriasis, burns, skin wounds and other skin disorders, in addition to: arthritis, back pain, bursitis, Carpal Tunnel Syndrome, headaches, neck pain, repetitive stress injuries, TMJ pain, tendonitis, sprains, swelling; and
the approximate output wavelength of a blue-light pulsating and/or oscillating and/or stationary laser and/or LED is in the range of, but not limited to 400 to 420 nanometers, for the treatment of acne bacteria, enlarged pores, oily skin, stretch marks, wound healing, folliculitis, scar tissue, excessive perspiration, viral warts, psoriasis, precancerous solar keratosis or skin lesions, aging, wrinkled or sun damaged skin, skin ulcers, cellulite, skin cancer and damaged DNA primarily due to environmental exposure to the sun, normalizing melanin pigmentation, and stimulating collagen; and
the approximate output wavelength of a blue-light pulsating and/or oscillating and/or stationary laser and/or LED is in the range of, but not limited to 390 to 490 nanometers, for treatment of the molecular clocks of cells, and the biological and Circadian Rhythm clocks of humans which cause: insomnia, jet lag, cancers, immune dysfunction disorders, space flight related, prolonged underwater habitation, and to counteract the damaging effects of sun exposure, or after an accidental sunburn, or other types of affected skin; and
the approximate output wavelength of a blue-light pulsating and/or oscillating and/or stationary laser and/or LED is in the range of, but not limited to 300 to 390 nanometers, for treatment of DNA damage by reproducing DNA pyrimidine dimers; and
the approximate output wavelength of a green-light pulsating and/or oscillating and/or stationary laser and/or LED is in the range of, but not limited to 491 to 574 nanometers, for treatment of third degree burns, and promotion of healing open wounds;
whereby the skin of the individual is exposed during application of energy from the laser beams/LED's to the affected skin areas.

7. A device, as in claim 6, further comprising:
the additional step of providing independent means such as a manual master switch to activate or deactivate the lasers and/or LED's;
whereby the individual can selectively emit laser/LED energy in the form of stationary, pulsating and/or oscillating light beams.

8. A device as in claim 7, further comprising:
either a manual or automatic switch for each row of lasers and/or LED's to activate or deactivate each row, as the battery power begins to expire; and
a switch that turns off the device after a prescribed amount of time used;
whereby the individual can extend battery life and maximize the remaining power.

9. A wearable laser hair, scalp, and skin treatment device as in claim 8, further comprising:
a housing and an adjustable one size fits all headband; and
laser assemblies secured within or to said housing, preferably by means of multiple tracks for outputting laser beams and/or LED's in a parallel and/or staggered and/or overlapping formation;
whereby as much of the scalp as possible will be exposed to the light.

10. A device as in claim 9, with a housing further including:
a battery pack; and
a solar cell(s) assembly positioned on top of the exterior of the housing further comprising:
a manual switch for solar use, battery use, external power source use, or solar charging of the battery;
whereby the individual can selectively choose the power source he intends on using.

11. A device as in claim 10, with a lightweight housing further including:
a reflective mirrored interior, such as chrome, silver, or any other reflective surface;
whereby any stray laser light can be reflected back to the scalp.

12. A device, as in claim 11, wherein:
the housing is in the form of, but not limited to a hat, helmet, Skull cap, or other wearable device on the head; and
composed of plastic, metal, cloth, fiberglass, graphite, or any other lightweight material suitable; and
individual laser diodes which can be inserted into the outer ear, irradiating through the middle ear, and into the inner ear;
whereby the device can be portable, wearable, and hands free.

13. A portable, table resting device known as “the support base” comprising:
a hollow box, further comprising;
a mirrored interior bottom and walls;
a clear glass top;
a support arm for the device in claim 1;
whereby the individual could slide the head device onto the support base via the support arm, and use the support base to treat other parts of the body with the laser/LED treatments.

14. A device as in claim 1, may be used as a hair regrowth system in conjunction with:
a topical Minoxidil-Finasteride solution, suspension, or emulsion, alone, or in combination, or in other forms common or known to those skilled in art of topical and oral delivery in the amount of, but not limited to:
a 2 oz. topical mixture containing 5% Minoxidil with 25 mg. of Finasteride in a solution, suspension, or emulsion, or combination thereof, to be applied topically to the male scalp at a rate of, but not limited to, 1 ml. per application, twice daily, with a physician's prescription; and
a topical Ketoconazole shampoo, solution, suspension, or emulsion, alone, or in combination, or in other forms common or known to those skilled in art of topical and oral delivery in the amount of, but not limited to:
a 2% topical mixture,
whereby the new hair growth can be expedited with multiple hair re-growth techniques.

15. A portable, folding, body skin treatment chamber and system, comprising:
a folding lightweight box, large enough to house a seated individual, further comprising:
easily changeable and adjustable laser assemblies and/or Light Emitting Diodes (LED's) attached to the walls which snap or screw into place, further comprising:
a laser beam generator(s) and/or LED generators; and
a power supply for supplying power to the laser beam generator(s) which may include but are not limited to:
batteries, solar cells, Alternate Current, car adaptor or other power sources; and
means to direct at least one laser beam and/or LED toward the skin from the laser assembly; and
a clear chair; and
a mirrored interior; and
a top that folds down around one's neck, completely covering one's body, except the head;
whereby the entire body of the individual can be exposed during application of energy from the laser beams to the skin.

16. A device as in claim 2 wherein:
each unique laser beam assembly and/or LED contains a snap in, or screw in connector, similar to that of a light bulb;
whereby the laser and/or LED modules can be easily changed in the case of a “burn out” or in the case of changing the wavelength being used.

17. A method for using the device of claim 9 by applying laser/LED energy to an individual's scalp and/or ears and/or gums, including the steps of:
applying laser energy to the scalp of an individual by wearing a laser and/or LED generator(s) across the individual's scalp in a hat-like device; and
applying laser energy to the ears of an individual by wearing a laser and/or LED generator(s) in a hat-like device that may contain snap, or screw in, or adherence with an adhesive such as Velcro/tape on, ear flaps; and
applying laser energy to the gums of an individual by wearing a laser and/or LED generator(s) in a hat-like device that may contain a snap, or screw on goose neck adjustable adapter; and
maximizing the amount of laser energy applied to the scalp, gums, and/or ears of the individual by using stationary, pulsating, and/or oscillating line, elliptical, and/or other shaped generators, spaced parallel or overlapping with each other;
whereby the entire head and/or middle and inner ears are encompassed with laser light.

18. A method as in claim 17, in another embodiment, including the additional step of:
further extending the surface area covered by the laser energy by splitting each single laser beam into multiple, substantially parallel, laser beams with a beam splitter;
whereby a large total area of scalp can be irradiated by each single laser beam.

19. A method as in claim 18, including the additional step of:
stationary, pulsating, and/or oscillating the laser beams;
whereby a higher, longer lasting light output power can be obtained.

20. A method for using the device of claim 6, for:
reducing hair loss in men and women;
reducing and eliminating dandruff in men and women;
treating loss of hair color in men and women;
increasing hair tensile strength in men and women;
stimulating new hair growth in men and women;
stimulating cellular repair in the middle and inner ear for hearing restoration, Tinnitus, and Vertigo in men and women;
stimulating cellular repair of gum tissue in men and women;
stimulating collagen production in men and women;
reducing and eliminating eczema in men and women;
reducing and eliminating psoriasis in men and women;
reducing and eliminating aging and wrinkles in men and women;
reducing and eliminating acne in men and women;
reducing and eliminating enlarged pores in men and women;
reducing and eliminating skin cancers in men and women;
reducing and eliminating stretch marks in men and women;
reducing and eliminating folliculitis in men and women;
reducing and eliminating excessive perspiration in men and women;
reducing and eliminating viral warts in men and women;
reducing and eliminating precancerous solar keratosis in men and women;
reducing and eliminating sun damaged skin in men and women;
reducing and eliminating skin ulcers in men and women;
reducing and eliminating cellulite in men and women;
reducing and eliminating damaged DNA in men and women due to environmental exposure to the sun and sunburn;
reducing scars in men and women;
treating Arthritis in men and women;
treating Bursitis in men and women;
treating Carpal Tunnel Syndrome in men and women;
treating headaches and neck pain in men and women;
treating tendonitis in men and women;
treating sprains in men and women;
treating swelling in men and women;
treating repetitive stress injuries in men and women;
treating TMJ pain in men and women;
treating biological and Circadian Rhythm clocks which lead to insomnia, jet lag, and immune dysfunction disorders in men and women; and
treating burns and skin wounds in men and women;
whereby these problems can be successfully treated and alleviated.
